(19) United States
(12) Patent Application Publication
(10) Pub. No.: US 2026/0207645 A1
(43) Pub. Date: Jul. 23, 2026

(54) USE OF INTEGRIN A5B1 AS TARGET IN SCREENING OF DRUGS FOR PREVENTING AND/OR TREATING BONE LOSS

(71) Applicant: The Hong Kong Polytechnic University, Hong Kong (CN)

(72) Inventors: Chui Wa Christina POON, Hong Kong (CN); Man Sau WONG, Hong Kong (CN)

(21) Appl. No.: 19/157,735

(22) PCT Filed: Feb. 1, 2024
(86) PCT No.: PCT/CN2024/075172

§ 371 Date: Aug. 19, 2025

(30) Foreign Application Priority Data
Apr. 11, 2023 (CN) 202310380869.2

(51) Int. Cl. A61K 31/7048; A61K 31/352; A61P 19/08; G01N 33/68

(52) U.S. Cl. CPC A61K 31/7048; A61K 31/352; A61P 19/08; G01N 33/68; G01N 2333/7055; G01N 2500/04

(57) ABSTRACT

The present application relates to the technical field of medicine. Provided is the use of integrin α5β1 as a target in the screening of drugs for preventing and/or treating bone loss. 8-isoprenyl flavone glycoside can replace a ligand fibronectin on cell surface receptor integrin α5β1, thereby achieving the binding of 8-isoprenyl flavone glycoside to the integrin α5β1, and thus improving the osteogenesis ability and the cell adhesion. Therefore, by using integrin α5β1 as a target of a drug for preventing and/or treating bone loss, and by means of the binding of 8-isoprenyl flavone glycoside to integrin α5β1, the activities of ALP, RhoA and Rac1 in bone marrow stromal cells can be significantly improved, thereby promoting the osteogenesis and the cell adhesion, promoting the development of drugs for bone loss, and providing a new strategy and routine for the treatment of bone loss diseases.

This application claims priority to the Chinese patent application filed with the China Patent Office on Apr. 11, 2023, with the application No. 202310380869.2 and the title “Use of integrin α5β1 as target in screening drugs for preventing and/or treating bone loss”, the content of which is incorporated herein by reference in its entirety.

### TECHNIAL FIELD

The present application relates to technical field of medicine, and particularly relates to the use of integrin α5β1 as target in screening for drugs for preventing and/or treating bone loss.

### BACKGROUND

With the development of space technology, astronauts are spending increasingly longer period in space stations. Bone loss and osteoporosis caused by microgravity have become important factors compromising physical health of astronauts and successful progress of space missions. Many doctors believe that bone loss is a precursor to osteoporosis, and that the primary cause for bone loss is decreased function of osteoblasts. Many studies have reported the effect of microgravity on different bone cell types. However, the specific mechanisms through which microgravity regulates and induces changes in bone tissue remain poorly understood. Although artificial bones have high osteoconductivity, they are not osteoinductive and thus cannot fundamentally address microgravity-induced bone loss. Currently, growth factors such as bone morphogenetic protein (BMP-2) are commonly incorporated into artificial bones to increase their osteoinductivity. However, the clinical application of bone morphogenetic protein in artificial bones remains limited due to its rapid in vivo degradation and high cost.

Isoprenyl flavonoids have demonstrated estrogen-mimetic properties, and are used to prevent postmenopausal osteoporosis. Studies have shown that the 8-isoprenylsubstituent at ring A of flavonoids could significantly affect the osteogenic activity, suggesting isoprenyl flavonoids with C-8 isoprenyl may represent a unique class of flavonoids with a higher osteogenic activity. For example, 8-prenylgenistein (8-PG) was more potent than genistein in increasing osteoblastic differentiation and mineralization in UMR 106 cells. Icariin isolated from Herba Epimedii (HEP), was demonstrated to have osteoprotective effects against microgravity. The safe, inexpensive potential of 8-isoprenyl flavone make them potential drugs for the treatment of bone loss, as they impart anti-osteoporotic, osteogenic, chondrogenic and angiogenic effects. However, the cellular targets of 8-isoprenyl flavonoids have not been elucidated.

Thus, there is a need to identify the precise therapeutic targets to address the limitations of above-mentioned therapy, thereby offering an effective strategy for the prevention or treatment of these diseases.

### Technical Problem

An object of the present application is to use integrin α5β1 as a target in screening for drugs for preventing and/or treating bone loss. This aims to address the problem of unclear therapeutic target for microgravity-induced bone loss, which in turn leads to unsatisfactory therapeutic effects for bone loss.

### Technical Solutions

In order to achieve the above object, the present application adopts the following technical solutions:

In a first aspect, provided in the present application is the use of integrin α5β1 as a target in screening for drugs for preventing and/or treating bone loss.

In a second aspect, provided in the present application is the use of an 8-isoprenyl flavone glucoside as an integrin α5β1 ligand in the manufacture of a medicament for preventing and/or treating bone loss.

In a third aspect, provided in the present application is a medicament for preventing and/or treating bone loss, comprising an effective dose of an integrin α5β1 ligand.

### BENEFICAIL EFFECT

The present invention demonstrates through experimental analysis that 8-isoprenyl flavone glucosides can replace fibronectin—the sole ligand for the cell surface receptor integrin α5β1—and bind to integrin α5β1, thereby enhancing osteogenic capacity and cell adhesion. Therefore, by using integrin α5β1 as a target of a drug for preventing and/or treating bone loss, and by means of the binding of 8-isoprenyl flavone glycoside to integrin α5β1, the activities of ALP, RhoA and Rac1 in bone marrow stromal cells (BMSCs) can be significantly improved, thereby promoting the osteogenesis and cell adhesion, promoting the development of drugs for bone loss, and providing a new strategy and routine for the treatment of bone loss diseases.

### BRIEF DESCRIPTION OF THE DRAWINGS

To more clearly illustrate the technical solutions in the embodiments of the present application, the drawings required for describing the embodiments or the prior art are briefly introduced below. It is evident that the drawings in the following description are merely some embodiments of the present application, and other drawings can be derived from these drawings by those of ordinary skill in the art without making creative efforts.

FIG. 1 is a schematic diagram of a computerized docking structure of icariin binding to integrin α5β1, as provided in Example 1 of the present application;

FIG. 2 is a schematic diagram of a computerized overall structure of icariin binding to integrin α5β1, as provided in Example 1 of the present application;

FIG. 3 is a schematic diagram of the best pose of icariin binding to integrin α5β1, as provided in Example 1 of the present application;

FIG. 4 is a schematic diagram of the results of a competitive solid-phase binding enzyme-linked immunosorbent assay, as provided in Example 2 of the present application;

FIG. 5 is a confocal microscopic image of cells under normal gravity and microgravity conditions, as provided in Example 3 of the present application;

FIG. 6 is a quantitative analysis of ALP (a), RhoA (b), and Rac1 (c) activities under normal gravity and microgravity conditions, as provided in Example 3 of the present application.

### DETAILED DESCRIPTION

The present application will be described in further detail below in combination with embodiments in order to make the technical problems to be solved, technical solutions, and beneficial effects of the present application clearly understood. It is to be understood that the specific embodiments described herein are merely for illustration of the present application and are not intended to limit the present application.

In the present application, the term “and/or”, which describes an association relationship between associated objects, denotes that three kinds of relationships may exist. For example, “A and/or B” may mean that only A exists, both A and B exist, or only B exists, wherein A, B may be singular or plural. The symbol “/” generally indicates that the contextual object is an “OR” relationship.

In the present application, “at least one” refers to one or more and “a plurality of” refers to two or more. “At least one of the following” or similar expressions thereof refer to any combination of these items, including any combination of single or plural items. For example, “at least one of a, b, or c” or “at least one of a, b, and c” may both mean: a, b, c, a-b (i.e. a and b), a-c, b-c, or a-b-c, wherein a, b, and c may be single or multiple, respectively.

It should be understood that in various embodiments of the present application, the numerical order of the aforementioned processes does not imply the sequence of execution. Some or all of the steps may be executed in parallel or sequentially, and the execution order of each process shall be determined based on its function and inherent logic, and shall not constitute any limitation on the implementation process of the embodiments of the present application.

The terminology used in the embodiments of the present application is for the purpose of describing particular embodiments only and is not intended to limit the present application. The singular forms “a,” “an,” “said,” and “the” as used in the embodiments of the present application and the appended claims are also intended to include the plural forms unless the context clearly indicates otherwise.

The weight of a relevant ingredient mentioned in the description of the embodiments of the present application may refer not only to the specific content of each component but may also to the proportional relationship between the weights of the components. Therefore, any proportional scaling up or down of the content of the relevant components according to the description of the embodiments of the present application falls within the disclosure of scope of the description of the embodiments of the present application. Specifically, the masses described in the description of the embodiments of the present application may be mass units known in the chemical field, such as μg, mg, g, kg, and the like.

The terms “first” and “second” are used solely for descriptive purposes to distinguish between objects (such as substances), and shall not be understood as indicating or implying relative importance, or as implicitly specifying the quantity of technical features referred to. For example, a first XX may also be referred to as a second XX, and similarly, a second XX may also be referred to as a first XX, without departing from the scope of the embodiments of the present application. Thus, a feature defined with “first” or “second” may explicitly or implicitly include one or more of instances of that feature.

The term “8-PN” is an abbreviation for “8-prenylnaringenin”. The term “8-PG derivative” is an abbreviation for “8-prenyl genistein derivative”. “BMSCs” is an abbreviation for “bone marrow stromal cells”. “F-actin” represents fibrous actin.

A first aspect of an embodiment of the present application provides the use of integrin α5β1 as a target in screening for drugs for preventing and/or treating bone loss.

The present invention demonstrates through experimental analysis that 8-isoprenyl flavone glucosides can replace fibronectin—the ligand for the cell surface receptor integrin α5β1—and bind to integrin α5β1, thereby enhancing osteogenic capacity and cell adhesion. Therefore, by using integrin α5β1 as a target of a drug for preventing and/or treating bone loss, and by means of the binding of 8-isoprenyl flavone glycoside to integrin α5β1, the activities of ALP, RhoA and Rac1 in BMSCs can be significantly improved, thereby promoting the osteogenesis and cell adhesion, promoting the development of drugs for bone loss, and providing a new strategy and routine for the treatment of bone loss diseases.

A second aspect of the present application provides the use of an 8-isoprenyl flavone glucosides as an integrin α5β1 ligand in the manufacture of a medicament for preventing and/or treating bone loss.

In some embodiments, the medicament comprises a drug for increasing the activities of ALP, RhoA, and Rac1. When 8-isoprenyl flavone glucosides bind to integrin α5β1, the activities of cytoskeletal proteins ALP, RhoA, and Rac1 can be significantly increased, thereby promoting osteogenesis and cell adhesion, achieving the prevention and/or inhibition of bone loss, and maintaining bone health.

In some embodiments, the 8-isoprenyl flavone glucosides comprise at least one of icariin, 8-prenylnaringenin, and 8-prenylgenistein. 8-isoprenyl flavone glucosides can replace fibronectin—the ligand of cell surface receptor integrin α5β1—and bind to integrin α5β1. The IC₅₀ values of icariin, 8-prenylnaringenin, and 8-prenylgenistein in competing with fibronectin are 9.82±3.31 nM, 7.6±3.92 nM, and 0.39±0.28 μM, respectively. At this dosage, icariin, 8-prenylnaringenin, and 8-prenylgenistein can replace fibronectin—the only ligand of integrin α5β1—to bind to integrin α5β1 and improve osteogenesis and cell adhesion.

Specifically, the structural formula of the 8-isoprenyl flavone glucoside is shown in formula (I), wherein R₁, R₂, R₃, and R₄ can be flexibly selected as needed.

For example, R₁, R₂, R₃, and R₄ are each independently selected from C₁-C₃₀ linear or branched alkyl groups.

Specifically, the structural formula of icariin is shown in formula (II).

Specifically, the structural formula of 8-prenylnaringenin is shown in formula (III).

Specifically, the structural formula of 8-prenylgenistein is shown in formula (IV).

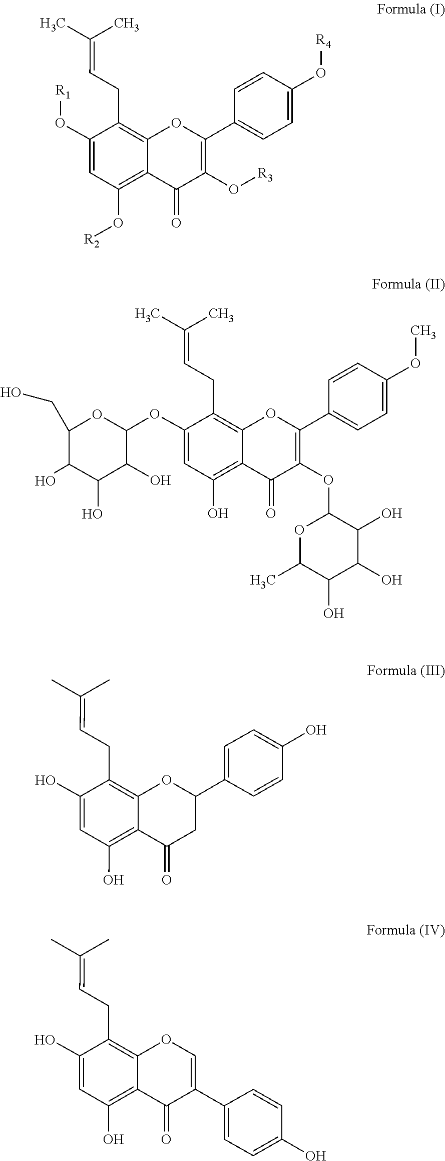

In some embodiments, bone loss includes microgravity-induced bone loss. The microgravity environment inhibits the differentiation of BMSCs into osteoblasts. In this environment, the actin filaments of BMSCs undergo depolymerization, which impairs osteogenic and cell adhesion ability, thereby causing bone loss. 8-isoprenylflavone glucosides can replace fibronectin (the ligand of cell surface receptor integrin α5β1) and bind to integrin α5β1, thereby improving the impaired osteogenic capacity and the cell adhesion caused by microgravity.

A third aspect of the present application provides a drug for preventing and/or treating bone loss, comprising an effective dose of an integrin α5β1 ligand. That is, the ingredient(s) that exert(s) an osteogenic effect in the medicament for preventing and/or treating bone loss comprise(s) at least an integrin α5β1 ligand.

In some embodiments, the 8-isoprenylflavone glucosides comprise at least one of icariin, 8-prenylnaringenin, and 8-prenylgenistein.

In some embodiments, the medicament uses an integrin α5β1 ligand as the sole active ingredient for preventing and/or treating bone loss.

In some embodiments, the medicament uses an integrin α5β1 ligand and other osteogenic agents as active ingredients for preventing and/or treating bone loss.

In some embodiments, pharmaceutically acceptable excipients are also included. Pharmaceutically acceptable excipients refer to any excipient known to those skilled in the art that is suitable for a specific administration mode, ensuring the convenient formulation and clinical application of the prepared medicament for preventing and/or treating bone loss.

Specifically, the pharmaceutically acceptable excipients may include one or more pharmaceutically acceptable carriers, solvents, excipients, buffers, lubricants, and binders that are compatible with the active ingredients of the embodiments of the present application. Among other things, the excipients should be non-toxic, not interfere with or impair the efficacy of the active ingredient of the embodiment of the present application, and the excipients can be flexibly selected based on the specific dosage form of the present application.

For example, the carriers include, but are not limited to, sugars such as lactose, glucose, and sucrose. The binders include, but are not limited to, starches such as corn starch and potato starch, cellulose and its derivatives such as sodium carboxymethyl cellulose, ethyl cellulose, and cellulose acetate, etc. The excipients include, but are not limited to, cocoa butter, suppository waxes, oils such as peanut oil, cottonseed oil, safflower oil, sesame oil, olive oil, corn oil, and soybean oil, glycols such as propylene glycol, esters such as ethyl oleate and ethyl laurate, and agar, etc. The buffers include, but are not limited to, at least one of magnesium hydroxide, aluminum hydroxide, alginic acid, pyrogen-free water, isotonic saline, Ringer's solution, ethanol, and phosphate buffer solution, etc. The lubricants include, but are not limited to, sodium lauryl sulfate and magnesium stearate, and the like.

In some embodiments, coloring agents, release agents, coating agents, sweetening agents, flavoring agents, aromatic agents, preservatives, and antioxidants can also be employed as excipients as determined by those of skill in the art. Specifically, diluents are added primarily to enhance the weight and bulk of the drug to facilitate molding and dosage division. Coating agents and colorants are added to improve the appearance of tablets, enhance the stability of medicament, mask unpleasant odor of medicament, and alter the appearance of granules.

For example, the coating agent is selected from, but not limited to, at least one of acrylic resin, hypromellose, povidone, and cellulose acetate phthalate. The coloring agent is selected from, but not limited to, at least one of titanium dioxide, Sunset Yellow, and methylene blue.

In some embodiments, the dosage form of the medicament for preventing and/or treating bone loss includes at least one of solutions, pills, tablets, capsules, powders, pastes, aerosols, and patches. However, the dosage form of the medicament for preventing and/or treating bone loss in the embodiments of the present application is not limited thereto, and other achievable dosage forms fall within the protection scope of the present application.

The invention will be further described in combination with specific examples.

### Example 1

In this example of the present application, taking icariin as an example, computerized molecular docking was performed between icariin and integrin α5β1.

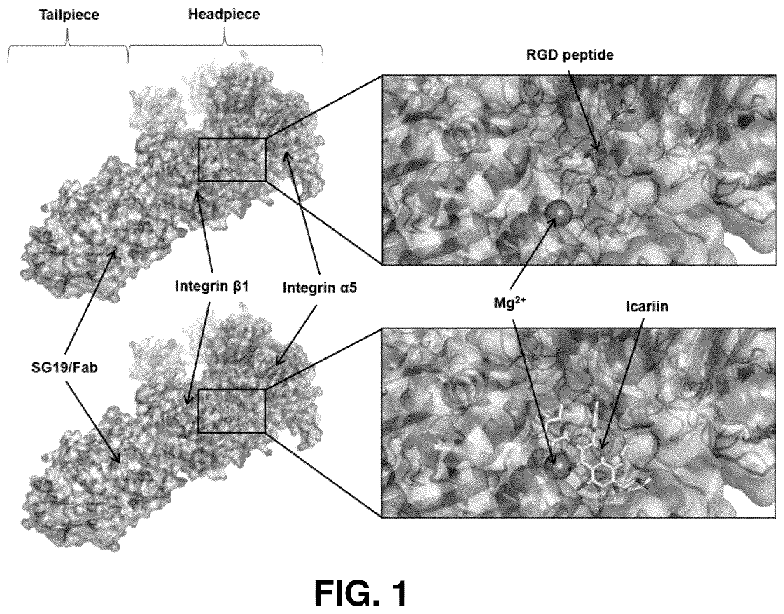

Integrin α5β1 is a cell adhesion receptor in bone cells, and Arg-gly-asp (RGD) peptide sequence is the integrin recognition motif in fibronectin. To gain insight into the interaction of icariin on integrin α5β1, computerized molecular docking was applied to perform automated docking calculations on the crystal structures of integrin α5β1 in complex with RGD peptide or icariin respectively. Computational models for the interactions of these ligands with integrin α5β1 were generated and the results are shown in FIGS. 1-3.

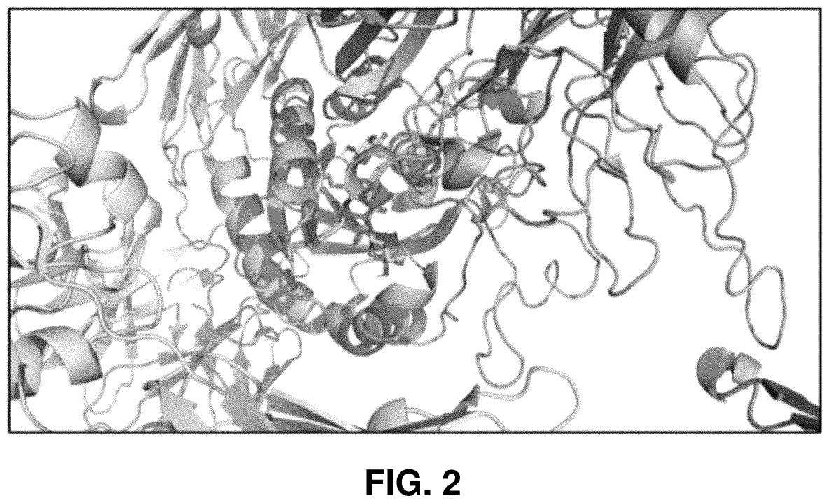

The docking structure of integrin α5β1 is shown in FIG. 1, and the ribbon representation of the overall structure is shown in FIG. 2. As can be seen from the figures, the best pose of icariin into integrin α5β1 binding site is at the headpiece with the magnesium ion (Mg²⁺) in the β1 subunit and the binding affinity is −7.9 kcal/mol. Magnesium ions can promote the ligand binding to integrin α5β1, suggesting its important role for the support of icariin binding on the β1 binding site.

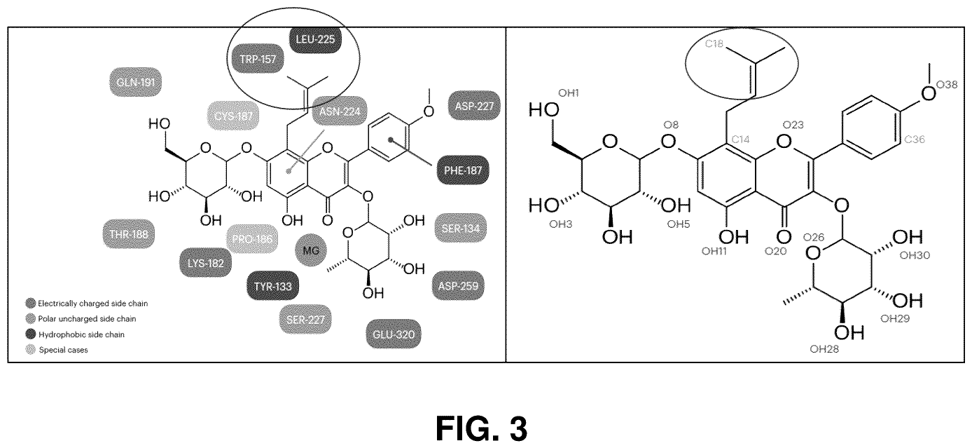

As can be seen from FIG. 3, the best pose of icariin into integrin α5β1 involves the binding of the isoprenyl group of icariin to residue Trp-157 in the α5 subunit and residue Leu-225 in the β1 subunit of the integrin receptor. Specifically, the parameters of the binding of icariin to integrin α5β1 are shown in below Table 1. It can be known that the 8-isoprenyl group is a key factor influencing the binding of icariin to integrin α5β1. This also indicates that icariin exhibits higher bone-protective activity and stronger osteogenic activity than non-isoprenylated icariin.

TABLE 1

| | Binding distance | | | Receptor |
|---|---|---|---|---|
| Position | (nm) | Atom | Residue | subunit |
| C18 | 3.45 | NE1 | Trp-157 | α5 |
| C18 | 3.91 | CD1 | Leu-225 | β1 |
| O20 | 2.44 | MG | | |

### Example 2

To gain insight into the interaction between 8-isoprenylflavone glucosides and integrin α5β1, soluble integrin and coating extracellular matrix protein (fibronectin) were employed to determine α5β1 integrin selectivity through competitive solid-phase binding enzyme-linked immunosorbent assay (ELISA).

The competitive solid-phase binding enzyme-linked immunosorbent assay can be performed with reference to the method described in Frank A. O. et al. (2010) Doi: 10.1002/annie.201004363. Specifically, 96-well ELISA plates (BRAND, Germany) were coated overnight at 4° C. with fibronectin (0.50 μg/ml) (Sigma, USA) in a buffer solution (15 mM Na₂CO₃, 35 mM NaHCO₃, pH 9.6). Subsequently, the plates were washed three times with PBST buffer (137 mM NaCl, 2.7 mM KCl, 10 mM Na₂HPO₄, 2 mM KH₂PO₄, 0.01% Tween 20, pH 7.4) and blocked for one hour at room temperature with TSB buffer (20 mM Tris-HCl, 150 mM NaCl, 1 mM CaCl₂), 1 mM MgCl₂, 1 mM MnCl₂, pH 7.5, 1% BSA). Soluble integrin α5β1 (1.0 μg/ml) (R&D Systems, MN, USA) and a serial dilution of 8-isoprenyl flavone glucosides (10⁻¹³-10⁻⁵ M) in TSB buffer were incubated in the coated wells for 1 hour at room temperature. The plates were then rinsed three times with PBST buffer. The primary antibody (CD49e) (Pharmingen, BD Bioscience, USA) was incubated at 1.0 μg/mL in TSB (1:500 dilution) for 1 hour at room temperature. After washing three times with PBST, the secondary antibody (anti-mouse-HRP conjugate) (Invitrogen, USA) was applied at 2.0 μg/ml in TSB buffer (1:385 dilution) for 1 h at room temperature. After this treatment, the plates were washed three times and detection of HRP was performed using TMB substrate solution (3,3,5,5′-tetramethylethylenediamine) (Thermo Scientific, USA) and 2 M H₂SO₄ was used to stop the reaction. The absorbance was measured at 450 nm by Varioskan LUX multimode microplate reader (Thermo Scientific, USA), and every concentration was analyzed by duplicate to obtain inhibition curves, and the IC₅₀ values were calculated.

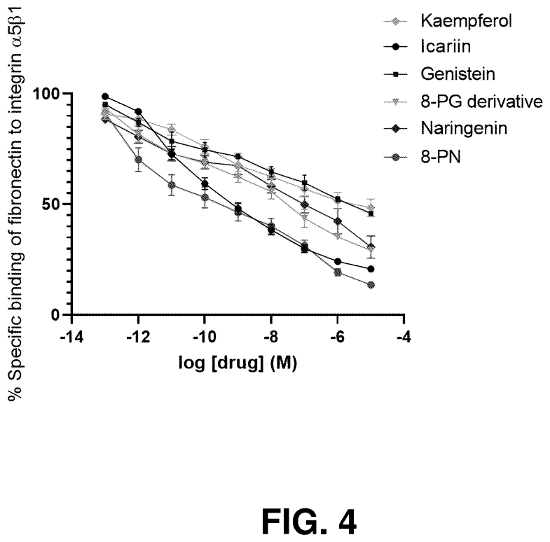

In this example, competitive solid-phase binding ELISA assays were performed on icariin, 8-PN, 8-PG derivatives, and non-isoprenylated flavonoids (kaempferol, naringenin, and genistein), and the results are shown in FIG. 4. As can be seen from FIG. 4, the computerized molecular docking results correlate nicely with the competitive solid-phase binding enzyme-linked immunosorbent assay (ELISA) results. The IC₅₀ values of icariin, 8-PN, 8-PG derivatives for competing with the specific ligand, fibronectin, bound on integrin α5β1 are 9.82±3.31 nM, 7.6±3.92 nM, and 0.39±0.28 μM, respectively, which are significantly lower than the IC₅₀ values of the non-isoprenylated flavonoids kaempferol and naringenin. The lower IC₅₀ value of icariin and 8-PN demonstrated its higher potency on binding to integrin α5β1, suggesting the 8-isoprenyl containing 8-isoprenylflavone glucosides have stronger binding to integrin α5β1.

### Example 3

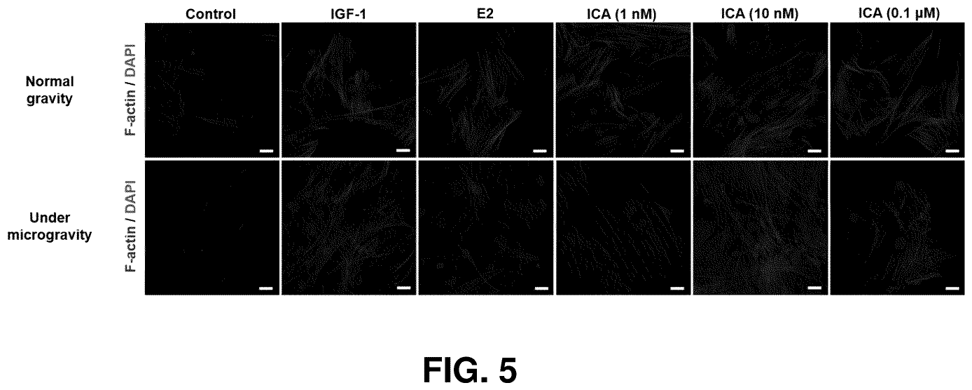

To demonstrate the effect of icariin on osteoblast differentiation, cell adhesion was tested under normal gravity and microgravity (mechanical unloading condition), respectively. Testing procedure: BMSCs were cultured in osteogenic medium. Icariin (ICA 1 nM, ICA 10 nM, and ICA 0.1 μM) and growth factors (IFG-1, E2) were added respectively, followed by microgravity treatment in a multi-directional gravity device for 3 days, and the F-actin microfilaments were labeled with Alexa Fluor™594 Phalloidin (red). The representative images were captured at mid-plane of the cells and visualized using a confocal laser scanning microscope (magnification: 400×, scale bar: 25 μm). Hoechst counter-staining (blue; DAPI) was applied for determination of single cells. The results of the responses of the actin cytoskeleton structure in differentiated BMSCs to icariin, IGF-1, and E2 are shown in FIG. 5.

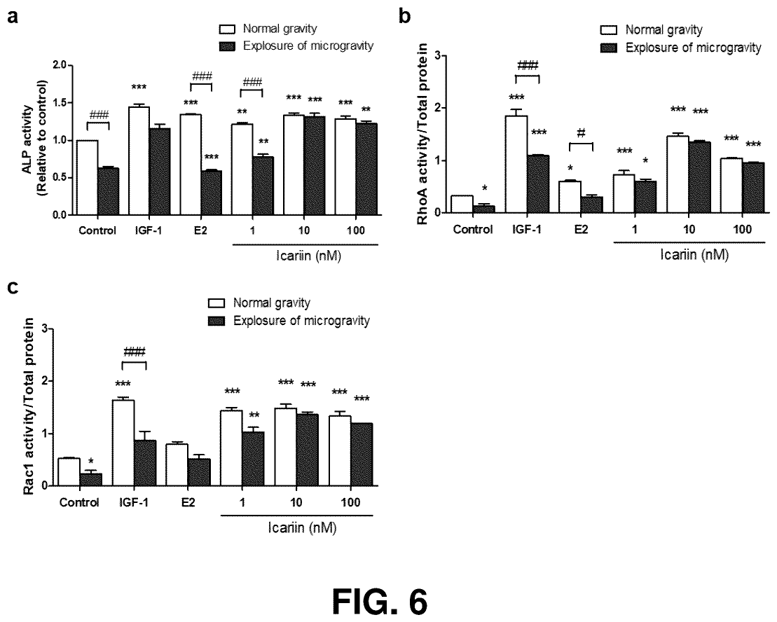

As can be seen from FIG. 5, the actin filaments of BMSCs in control became depolymerized and disrupted in dispersed distribution under microgravity, compared to the well-organized, thick actin filaments in cells under normal gravity. In addition, it can also be seen from FIG. 5 that only growth factor IGF-1 and icariin (10 nM and 0.1 μM) stimulated F-actin polymerization under microgravity, but not E2, which is consistent with the effects of IGF-1 and icariin on the inhibition of ALP activities in FIG. 6.

The quantitative analysis of the differentiation activity of BMSCs under normal gravity and microgravity is shown in FIG. 6, where FIG. 6a is the ALP activity analysis relative to the positive control group, FIG. 6b is the RhoA activity analysis in total protein, and FIG. 6c is the Rac1 activity analysis in total protein. The data in FIGS. 6a, 6b, and 6c are presented as mean values±SEM. Statistical analysis was performed using one-way ANOVA, *p<0.05, **p<0.01, ***p<0.001; versus control: *p<0.05, ###p<0.001 (n=3).

As can be seen from FIG. 6, the microgravity environment could significantly reduce the activities of ALP, RhoA, and Rac1 in differentiated BMSCs. Icariin at concentrations of 10 nM and 100 nM could restore the differentiation of BMSCs and exert similar bone-protective effects. Similar to the positive control IGF-1, icariin could significantly increase ALP, RhoA, and Rac1 activities in the cells to promote osteogenesis and improve cell adhesion.

The above-mentioned examples are merely preferred examples of the present application and not intended to limit the present application. Any modification, equivalent replacement or improvement made within the spirit and rule of the present application should be shall within the scope of protection of the present application.

1. A method of screening for drugs for preventing and/or treating bone loss comprising using integrin α5β1 as a target.

2. A method of preventing and/or treating bone loss comprising administering to a patient an 8-isoprenyl flavone glucoside as an integrin α5β1 ligand.

3. The method according to claim 2, characterized in that the medicament comprises an agent for increasing the activities of ALP, RhoA, and Rac1.

4. The method according to claim 2, characterized in that the 8-isoprenyl flavone glucoside comprises at least one of icariin, 8-prenylnaringenin, and 8-prenylgenistein.

5. The method according to claim 2, characterized in that the bone loss comprises microgravity-induced bone loss.

6. A medicament for preventing and/or treating bone loss, characterized in that the medicament comprises an effective dose of an integrin α5β1 ligand.

7. The medicament according to claim 6, characterized in that the integrin α5β1 ligand comprises an 8-isoprenyl flavone glucoside.

8. The medicament according to claim 7, characterized in that the medicament uses the integrin α5β1 ligand as the sole active ingredient for preventing and/or treating bone loss.

9. The medicament according to claim 7, characterized in that the medicament uses both the integrin α5β1 ligand and other osteogenic agents simultaneously as active ingredients for preventing and/or treating bone loss.

10. The medicament according to any one of claims 6-9, characterized in that the medicament further comprises a pharmaceutically acceptable excipient.
